(19) United States
(12) Patent Application Publication
(10) Pub. No.: US 2002/0001579 A1
(43) Pub. Date: Jan. 3, 2002

(54) Nonhuman helper-dependent virus vector

(76) Inventors: Moritz Hillgenberg, Berlin-Mitte (DE); Christian Hofmann, Berlin (DE); Peter Loser, Berlin (DE)

Correspondence Address: FULBRIGHT & JAWORSKI, LLP, 666 FIFTH AVE, NEW YORK, NY, 10103-3198, US

(21) Appl. No.: 09/785,563

(22) Filed: Feb. 16, 2001

(30) Foreign Application Priority Data
Feb. 16, 2000 (DE) 100 06 886.3

(51) Int. Cl. A61K 48/00; C12N 7/00; C12N 15/86

(52) U.S. Cl. 424/93.21; 435/235.1; 435/320.1; 435/457

(57) ABSTRACT

The invention relates to a nonhuman helper-dependent virus vector for transferring nucleic acid sequences. Areas of application are medicine, veterinary medicine, biotechnology and genetic engineering.

### DESCRIPTION

The invention relates to a nonhuman helper-dependent virus vector for the transfer of nucleic acid sequences. The areas of application are medicine, veterinary medicine, biotechnology and genetic engineering.

In recent years numerous methods and vectors for gene transfer have been developed with the aim of gene therapy or vaccination (review: Verma, M. I. and Somia, N. (1997). Nature 389, 239-242). The favorite vectors in these cases for gene transfer with the aim of gene therapy are, in particular, those derived from retroviruses, adeno-associated viruses (AAV) or human adenoviruses. These vector types have a wide range of cell types which can be infected efficiently and are therefore suitable for gene transfer into various tissues.

The so-called adenoviral vectors of the first generation have been intensively researched in the last decade as gene transfer vectors (review: Bramson, J. L. et al (1995). Curr. Op. Biotech. 6, 590-595). They were derived from the human adenovirus of serotype 5 and are deleted in the essential E1 region, often as well in the nonessential E3 region, making it possible to insert up to 8 KBp of foreign DNA into the viral genome. These vectors can be produced up to high titers in cells complementing the E1 deficiency, can be stored satisfactorily and mediate efficient gene transfer in vitro and in vivo. However, in vivo there is rapid loss of expression of the transgenes. In addition, in some cases extensive tissue toxicity has been observed after administration of high vector doses. The causes of both are at least in part immunological reactions to the viral genes remaining in the vector. Attempts have therefore been made in particular to take out further early viral genes, but no decisive improvement in in vivo gene transfer was achievable thereby.

Recently developed, entirely recombinant human adenovirus vectors (Kochanek, S. et al. (1996). Proc. Natl. Acad. Sci. USA 93; 5731-5736) showed in animal models efficient gene transfer with reduced toxicity and prolonged transgene expression (Morral, N. et al. (1998), Hum. Gen. Ther. 9: 2709-2716). However, since these vectors also have the normal envelope of the human adenovirus and, in the vector genome, the cis elements necessary for replication and packaging—the inverted terminal repeats (ITRs) and the packaging signal—these vectors still display two limitations which may be regarded as the main problems of human adenovirus vectors. The reasons for both are the human origin of the adenoviruses used and the wide distribution thereof in the human population: (1) the preexisting antibodies which are usually present lead to substantial neutralization of the vector. Current studies in animal models have shown that efficient gene transfer is possible when there are preexisting antibodies only on use of high vector doses (Nunes, F. A. et al. (1999), Hum. Gen. Ther. 10, 2515-2526), but this may lead to severe toxic side effects, inter alia including serious hematological side effects (Cichon, G. et al. (1999), J. Gene Med. 1, 360-371). (2) In addition there is the risk of coreplication of the recombinant vector in the event of natural infection with a wild-type adenovirus with unpredictable consequences. Current human adenovirus vectors can therefore be considered for use for human gene therapy to only a limited extent. One approach to overcoming the problem of preexisting antibodies against human adenoviruses was recently found through the development of adenoviral vectors of nonhuman origin. The viral genome in these vectors is substantially unchanged, and usually relatively small regions nonessential for virus propagation have been replaced by a transgene (Mittal, S. K. et al. (1995), J. Gen. Virol. 76, 93-102; Klonjkowski, B. et al. (1997), Hum. Gen. Ther. 8: 21032115; Michou, I. et al. (1999). J. Virol. 72, 1399-1410. These vectors have, however, an insertion capacity limited to a few KBp for each transgene, little research has been done on their biology in most cases, and to date they have not been characterized in particular in relation to their safety in the human body. Packaging signals of nonhuman adenoviruses have not to date been located.

One object of the present invention was thus to provide novel vectors for gene transfer, in which the disadvantages of previous vectors, in particular presence of a preexisting immune response in humans, limited foreign DNA capacity or/and possible side effects derived from viral genes which have to date been characterized only slightly or not at all, are at least partly eliminated.

A first aspect of the invention relates to a vector in particular for cloning viral genomes, for example genomes of nonhuman adenoviruses, and the use thereof for identifying and characterizing packaging sequences of the nonhuman adenoviruses, and for producing adenoviral gene transfer vectors. This cloning vector comprises

(a) a packaging sequence of a bacteriophage,

(b) at least one cloning site for inserting heterologous nucleic acid sequences,

(c) at least two cleavage sites for a restriction endonuclease which flank both sides of the cloning site (b),

(d) a bacterial origin of replication, and

(e) a bacterial selection marker gene.

The cloning vector comprises elements which permit propagation and selection in prokaryotic host organisms, in particular in Gram-negative bacteria such as, for example, E. coli, in particular a bacterial origin of replication, for example the ColE1 origin of replication from E. coli, and of a bacterial selection marker gene, in particular an antibiotic-resistance gene such as, for example, the ampicillin-resistance gene. The vector comprises a cloning site for inserting heterologous nucleic acid sequences, in particular heterologous nucleic acid sequences with a length of ≧10 kBp. The vector is preferably a bacteriophage vector or cosmid vector. It preferably comprises the bacteriophage packaging sequences from a lambdoid phage, in particular the cos element of phage lambda.

The restriction cleavage sites flanking both sides of the cloning site are preferably the cleavage sites for a restriction endonuclease with a recognition specificity of at least 8 base pairs, in particular the cleavage sites for a meganuclease such as, for example, I-SceI.

The cloning site is preferably a multiple cloning site, i.e. it comprises a plurality of preferably unique cleavage sites for restriction endonucleases.

It is possible to insert into the cloning site of the vector the genome of a nonhuman adenovirus, i.e. an adenovirus which naturally occurs in a nonhuman species which is selected, for example, from mammals and birds as listed in Russell, W. C. and Benkö, M. (1999), Adenoviruses (Adenoviridae): Animal viruses. In: Granoff, A. and Webster, R. G. (Eds): Encyclopedia of Virology, Academic Press, London, in particular adenoviruses from monkeys (SAV, various serotypes), goats (caprine, various serotypes), dogs (CAV, various serotypes), pigs (PAV, various serotypes), cattle (BAV, various serotypes), sheep (OAV1-6 and OAV287) and chickens (FAV, various serotypes) and EDS virus. The virus is preferably an adenovirus from sheep or cattle. Examples of suitable sheep adenoviruses are ovine mastadenoviruses or ovine atadenoviruses such as, for example, the OAV isolate 287, whose nucleotide sequence is indicated under Genbank Acc. No. U40398. Suitable bovine adenoviruses are bovine atadenoviruses or bovine mastadenoviruses. Particularly interesting in this connection are bovine and ovine atadenoviruses which are negative in the complement fixation assay.

The cloning vector of the invention can be used to produce partially deleted genomes of nonhuman adenoviruses. For this purpose, the viral genome inserted into the vector can be partially deleted by cleavage with suitable restriction endonucleases or combinations of restriction endonucleases. It is possible in this way to characterize the location of the viral packaging sequences in the genome.

In particular, the viral genome is inserted into the phage or cosmid vector of the invention in the cloning site, i.e. between the two cleavage sites for a meganuclease, and the infectivity of the viral genomes released therefrom by meganuclease digestion is determined after transfection into a packaging cell line. On the basis of the phage and cosmid vector containing the viral genome there is production of deletion mutants in which various parts of the viral genome are replaced by heterologous nucleic acids, for example by noncoding DNA, and a reporter gene cassette. The deletion mutants are transfected, after meganuclease digestion, into a packaging cell line which is subsequently infected with a helper virus and lyzed after occurrence of the cytopathic effect. The resulting lyzates are used to infect cells. Cells expressing the reporter gene contain a coreplicated and packaged helper-dependent nonhuman virus. It is possible in this way to localize the region of the viral genome which, besides the ITRs, must remain in the viral genome to produce infectious particles transducing the reporter gene (packaging sequence). Further deletions allow the location of the packaging sequence to be characterized more accurately by the same method.

A further aspect of the invention is thus a method for characterizing packaging sequences of nonhuman adenoviruses, where

(a) a predetermined region of the viral genome inserted into the cloning vector is deleted and replaced by a heterologous nucleic acid comprising a reporter gene cassette,

(b) the constructs produced in (a) are introduced into a helper system which provides the gene products necessary for replication and packaging of the nonhuman adenovirus,

(c) it is determined whether the system according to (b) results in nonhuman adenovirus particles containing the reporter gene, and

(d) steps (a), (b) and (c) are repeated, if necessary, until the cis sequences of the viral genome which are necessary besides the inverted terminal repeats (ITRs) for packaging (packaging sequences) are characterized.

The heterologous nucleic acid which replaces the deleted part of the viral genome is preferably similar in length to the deleted section and preferably comprises a genomic DNA, for example a noncoding genomic mammalian DNA, in particular a noncoding genomic human DNA. The heterologous nucleic acid additionally comprises a reporter gene cassette, i.e. a gene in expressible form which codes for a detectable polypeptide, for example the E. coli lac Z gene or a gene coding for a fluorescent protein, for example GFP.

The construct produced in this way is introduced into a helper system which provides the gene products necessary for replication and packaging of the nonhuman adenovirus. This helper system comprises a cell line which is permissive for the particular virus, for example the cell line CSL503 (Pye et al., Austr. Vet. J. 66 (1989), 231-232) for the sheep adenovirus OAV 287. The helper system additionally comprises a helper virus, for example the OAV 287 wild-type virus, which expresses the gene products necessary for packaging the deleted construct. Viral particles obtained from the helper system, for example after cell lysis, are subsequently investigated in a new infection cycle to find whether they express the reporter gene. It is possible in this way to localize the region of the viral genome which, besides the ITR regions, must remain in the viral genome to produce infectious particles transducing the reporter gene, i.e. the packaging sequence. The information obtained from such an experiment allows the location of the packaging sequence to be characterized accurately by producing further viral deletion variants. It was possible in this way, for example, to localize the location of the packaging sequence of the sheep adenovirus isolate OAV287 to the 5′-terminal 2.8 kBp or the 3′-terminal 1 kBp of the viral genome.

It is possible by characterizing the packaging sequence to produce novel nonhuman helper-dependent adenovirus vectors with partial or complete deletion of the coding viral genes, with retention of the cis elements necessary for replication and packaging, i.e. the ITR regions and the packaging sequence. This novel type of vector has crucial advantages compared with previously existing viral vector systems. Recombinant deletion variants can be produced from the entire range of nonhuman adenoviruses, so that it is possible to produce a diversity of suitable helper-dependent adenovirus vectors which satisfies the demands made on a vector for gene transfer, in particular for vaccination and gene therapy, and can be employed for corresponding applications in humans too.

It is possible to produce from the genome, inserted into the phage or cosmid vector of the invention, of the helper-dependent nonhuman virus, in which all the necessary cis elements for replication (ITRs) and packaging (packaging sequence) are present in the form of suitable viral 5′ and 3′ ends, while other regions of the virus are partly or completely deleted, a basic vector which in turn is suitable for producing a viral vector for gene transfer. A cloning site is introduced into the basic vector for insertion of heterologous DNA with or without size adjustment, for example a multiple cloning site or a larger piece of heterologous nonfunctional, preferably noncoding, DNA with suitable restriction cleavage sites between the viral 5′ and 3′ regions of the viral genome. Where appropriate, a reporter gene expression cassette can be introduced into one of the cleavage sites in the insertion site in order to make easier detection and titer determination possible on the helper-dependent nonhuman viruses to be generated. The basic vector is preferably a phage or cosmid vector, from which the viral genome of the gene transfer vector can be released by restriction cleavage, preferably by cleavage with a meganuclease.

A further aspect of the invention is a basic vector in particular for producing a viral vector for gene transfer, comprising

(a) a packaging sequence of a bacteriophage,

(b) viral sequences comprising two inverted terminal repeats (ITRs) and one or more packaging sequences of a nonhuman adenovirus, where the viral sequences are not able to bring about helper-independent viral replication and packaging in a permissive cell line,

(c) a cloning site with or without size adjustment for insertion of heterologous DNA and, where appropriate, a reporter gene cassette, which are located inside the viral sequences (b),

(d) at least two cleavage sites for a restriction endonuclease which flank both sides of the viral sequences (b),

(e) a bacterial origin of replication and

(f) a bacterial selection marker gene, where sequences (a), (e) and (f) are located outside the sequence region flanked by the cleavage sites (d).

The cloning site may be an insertion site with size adjustment, consisting of a noncoding DNA sequence with a length of 10 to 30 kBp. This noncoding DNA sequence may be a genomic sequence, for example a chromosomal DNA sequence from the human genome with at least two dual cleavage sites. On insertion of a heterologous nucleic acid sequence into the cloning site it is possible to delete different-sized parts of the DNA sequence present for size adjustment.

A helper-dependent nonhuman adenovirus vector with a transgene can be produced from this basic vector, for example by inserting an expression cassette for the transgene into the cloning site of the basic vector and then cutting the viral genome out of the basic vector.

A suitable transgene expression cassette can be introduced into the cloning site in the basic vector, for example by cosmid cloning. The resulting construct can, after meganuclease cleavage, be transfected in the helper system, and the helper-dependent nonhuman virus which is replicated and packaged by the helper system can be obtained by lysis of these cells. The helper-dependent nonhuman virus can be amplified by repeated introduction of the cell lyzate containing the helper-dependent nonhuman virus into the helper system.

A further aspect of the invention is thus a viral gene transfer vector comprising the envelope of a nonhuman adenovirus and genetic material which is packaged therein, which comprises

(a) viral sequences comprising two inverted terminal repeats (ITRs) and one or more packaging sequences of a nonhuman adenovirus,

(b) one or more nucleic acid sequences which code for peptides or polypeptides which are heterologous in relation to the nonhuman adenovirus, in operative linkage to expression control sequences and

(c) where appropriate a noncoding DNA, where the genetic material is not able to bring about helper-independent viral replication and packaging in a permissive cell line.

The viral sequences of the basic vector and of the gene transfer vector preferably comprise only the cis elements necessary for replication and packaging in the coat of the viral genome and are otherwise essentially free of functional nucleic acid sequences of the nonhuman adenovirus, in particular of nucleic acid sequences which code for gene products necessary for replication and packaging of the virus. The viral sequences particularly preferably comprise ≦10 kb and, in particular, ≦5 kb of nucleic acid sequences derived from the wild-type virus.

The genetic material of the gene transfer vector comprises nucleic acid sequences which code for peptides or polypeptides which are heterologous in relation to the nonhuman adenovirus, in operative linkage to expression control sequences, in particular to expression control sequences which permit expression in mammalian cells, for example in human cells. The expression control sequences may either be constitutively active in the desired target cell or be regulable. The expression control sequences may be of viral or cellular origin or comprise a combination of viral and cellular elements. Examples of suitable promoters are viral promoters, for example RSV promoter, CMV immediate early promoter/enhancer, SV40 promoter, or tissue-specific, and in this case especially liver-specific, promoters, for example the human albumin promoter (Ponder et al., Hum. Gene Ther. 2 (1991), 41-52) of the human α-1-antitrypsin promoter/enhancer (Shen et al., DNA 8 (1989), 101-108), the PEPCK promoter (Ponder et al., Hum. Gene Ther. 2 (1991), 41-52) or HBV-derived hybrid promoters, for example EIImCMV promoter (Löser et al., Biol. Chem. Hoppe-Seyler 377 (1996), 187-193). In addition, the expression-regulating sequences favorably comprise a polyadenylation signal, for example that of the bovine growth hormone gene (Goodwin & Rottman, J. Biol. Chem. 267 (1992); 16330-16334), that of the early transcription unit of SV40 (van den Hoff et al., Nucleic Acids Res. 21 (1993), 4987-4988) or that of the herpes simplex thymidine kinase gene (Schmidt et al., Mol. Cell. Biol. 10, (1990), 4406-4411).

The viral gene transfer vector can be employed for transferring heterologous nucleic acids into permissive cells, cell assemblages, organs and organisms, in particular for gene therapy or for vaccination. For the aim of gene therapy it is possible to use the genomic sequence or the cDNA of a gene whose product is lacking in the patient to be treated, occurs in nonphysiological quantities and is defective. It is also possible to employ a part of a genomic sequence which stretches over a mutation in the target gene and can undergo homologous recombination with the latter. For the aim of tumor gene therapy it is possible to employ various genes which bring about a slowing of growth or a killing of the tumor cells, where appropriate in combination with drugs or through immunostimulation. For the aim of vaccination it is possible to employ one or more optionally modified genes of a pathogenic organism against which immunization is to be achieved.

Preferred specific examples of transgenes are, for the aim of replacement gene therapy, genes for secreted serum factors (for example human coagulation factors IX (FIX) and VIII (FVIII), erythropoietin (Epo), α-1-antitrypsin (AAT)), and genes for proteins which might be employed for muscle disorders (for example dystrophin, utrophin), and the gene which is defective in Wilson's disease (ATP7B). Preferred specific transgenes for the aim of tumor therapy are tumor suppressor genes such as p16 or p53 (singly or in combination, for example p16/p53), genes for various interleukins (singly or in combination, for example IL2/IL7) and suicide genes, for example herpes simples virus type I thymidine kinase (HSV-TK).

The gene transfer vectors of the invention are nonhuman adenovirus vectors with an at least partly deleted viral genome. The adenovirus can—as has already been stated previously—originate from any nonhuman species. The adenovirus preferably originates from sheep or cattle, with particular preference being given to the sheep adenovirus OAV isolate 287.

In a preferred embodiment, the genetic material of the vector comprises (a) as viral sequences two inverted terminal repeats and one or more packaging sequences and (b) up to about 30 kBp of foreign DNA. The genetic material may additionally comprise one or more matrix attachment regions (MAR). Various matrix attachment regions can be employed, for example those located in an intron of the gene of human hypoxanthine-guanine phosphoribosyltransferase (HPRT) (Sykes et al., Mol. Gen. Genet. 212 (1988), 301-309) or those present in the human interferon-β gene (Piechazek et al., Nucleic Acids Res. 27 (1999), 426-428). Besides the coding transgene, the genetic material of the transfer vector comprises, for example, as backbone nucleic acids which do not encode proteins and may preferably originate from genomic mammalian DNA, in particular from genomic human DNA.

Yet a further aspect of the invention relates to the genetic material for packaging in a vector of the invention. The genetic material is a nucleic acid, preferably a DNA. The genetic material may be inserted for manipulation or/and amplification into a cloning vector as described previously, in particular between two meganuclease recognition sites in a cosmid vector.

Yet a further aspect of the invention is a system for producing the viral vectors of the invention. This system comprises

(a) the genetic material as defined previously,

(b) a helper system for providing the gene products necessary for replication and packaging of the vector, and

(c) where appropriate means for obtaining and purifying the vector.

The helper system preferably comprises a packaging cell line, i.e. a cell line which permits replication and packaging of the viral vector of the invention. In a preferred embodiment of the invention, the packaging cell line is a ready-made packaging cell line which provides, constitutively or inducibly, the gene products not encoded by the viral vector for the replication and packaging of the vector. The nucleic acids coding for these gene products can be introduced by transformation or transfection on suitable vectors, for example plasmids, or by means of homologous recombination, into the packaging cell line. The use of a ready-made packaging cell line has the advantage that no contamination of the viral vectors by helper viruses occurs. The packaging cell line is preferably a mammalian cell line of a species identical to the species from which the nonhuman viral vector is derived.

An alternative possibility is for the helper system also to comprise a packaging cell line in combination with a helper virus, in which case the helper virus wholly or partly provides the gene products necessary for replication and packaging of the vector. In this embodiment, the packaging cell line is chosen so that it provides the gene products necessary for replication and packaging of the vector only partly or not at all.

The helper virus is—as already stated previously—preferably an adenovirus from a nonhuman species, either a wild-type virus or a modified virus. It is expedient for the helper virus to be derived from the same species as the viral vector, or a related species. Thus, it is possible to use as helper viruses for example sheep adenoviruses such as, for example, ovine mastadenovirus or atadenoviruses, in particular the OAV isolate 287 or derivatives derived thereof. An alternative possibility is also to employ bovine adenoviruses, for example bovine atadenoviruses or bovine mastadenoviruses.

In order to increase the purity of the helper-dependent nonhuman virus vector, and to minimize or completely prevent possible contamination by helper viruses, it is preferred to use a helper system in which the packaging of the helper virus is disadvantaged compared to the helper-dependent nonhuman virus. This can take place, for example, by using a partially packaging-inhibited helper virus, in particular a helper virus whose packaging sequence is inactive, for example at least partially deleted. An alternative possibility is to use a packaging cell line which expresses a site-specific recombinase, in combination with a helper virus whose packaging signal is flanked by recognition sites for this site-specific recombinase, so that on coinfection of the packaging cell line with the helper virus and the genetic material of the viral vector the packaging signal of the helper virus is cut by the site-specific recombinase out of the viral genome, leading to packaging deficiency of the helper virus. If the gene for Cre recombinase is used as recombinase gene, the recombinase recognition sites are loxP sequences.

The viral vector can be obtained and purified from the helper cell line in a known manner (see, for example, Wold, W. M. S. (ed.): Adenovirus Methods and Protocols, Humana Press, Totowa, New Jersey, 1999). Preferably employed for this purpose are a cesium chloride density gradient centrifugation or/and an affinity chromatography separation.

The vector of the invention can be used to transfer genetic material into a target cell and, preferably, for expression of this genetic material in the target cell. The target cell is preferably a human cell. However, it is also possible to use nonhuman target cells, in particular nonhuman mammalian cells, for example for applications in veterinary medicine or in research. The gene transfer can take place in vitro, i.e. in cultivated cells, or else in vivo, i.e. in living organisms or specific tissues or organs of such organisms.

The vector is suitable for producing a composition for nucleic acid vaccination or a composition for gene therapy or, in particular, therapy of congenital or malignant disorders. Finally, the vector can also be employed for obtaining proteins by overexpression in cultivated cells.

The present invention makes a novel vector available for gene transfer which has crucial advantages compared with previously developed viral vectors such as retroviruses, human adenoviruses and other adeno-associated viruses. These include, in particular, the absence of a preexisting immune response in humans to the nonhuman vector, which makes efficient gene transfer possible with a low vector dose. In addition, because of the substantial or complete absence of viral gene expression in the target cells, the risk of unwanted side effects is minimized. For the same reason, the risk of immunological elimination of successfully transduced target cells is reduced, which is an important prerequisite for long-term expression of the transgene. The vector has a capacity for foreign DNA up to a size of about 30 kBp, which also makes it possible to insert large genomic sequences, a plurality of genes or complex regulatory elements for regulated or/and tissue-specific gene expression. It is possible in this way to diminish the risk of unwanted side effects due to aberrant sites of expression or/and strengths of expression.

The helper-dependent nonhuman adenovirus vectors of the invention thus make it possible to transfer tailored foreign DNA, in particular therapeutic DNA with a total size of up to about 30 kBp, into target cells while, at the same time, minimizing the risk of unwanted side effects after local or systemic administration in vivo. This provides an essential precondition for successful prevention of disorders caused by pathogens in animals and humans, and for therapy of genetic and malignant disorders in humans.

The preferred form of administration of the vector depends on the planned use. For muscle-directed gene transfer or transfer into a solid tumor, for example, local administration of the vector by intramuscular/intratumoral injection is to be preferred. Systemic introduction is possible for gene transfer into other target organs or tissues, for example by intraperitoneal, intraarterial or intravenous injection. Directed transfer into specific tissues or organs can in the latter case take place either by a natural or modified tropism of the vector for particular cell types or by selection of vessels which supply the tissue to be hit.

The dosage can be decided only after more detailed studies of the efficiency of gene transfer by the particular vector. Typically, 10⁷ to 10¹³, for example 10⁹ to 10¹¹, viral particles/kg of body weight will be employed. The exact dosage may, however, be modified depending on the nature of the vector, the nature and severity of the disorder and the mode of administration.

The invention is to be explained in more detail by the following figures and exemplary embodiments. These show:

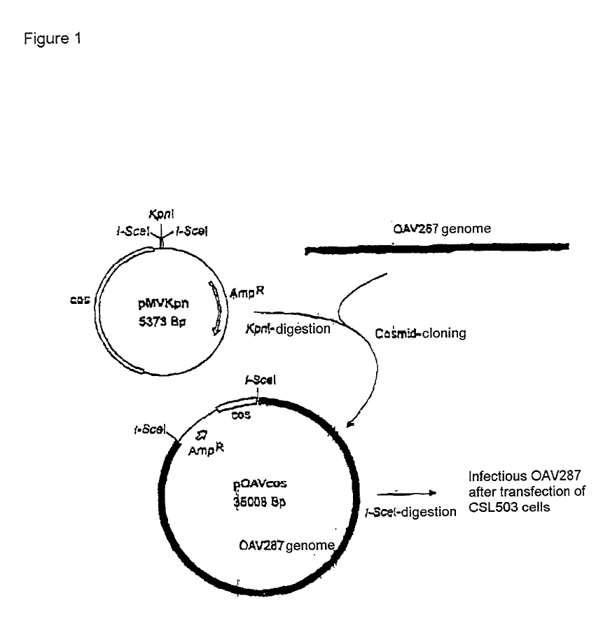

FIG. 1 a schematic representation of the cloning of the genome of the nonhuman adenovirus OAV 287 into the cosmid vector pMVKpn,

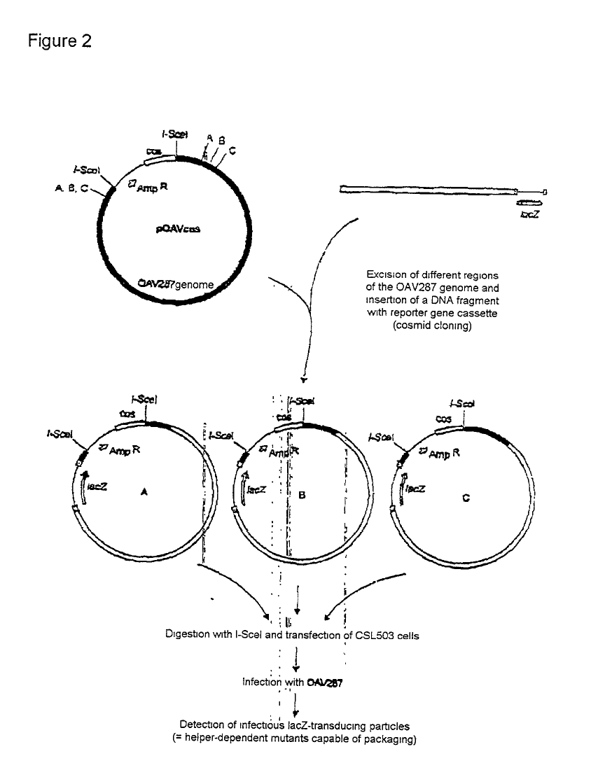

FIG. 2 a schematic representation of the experiments to localize the packaging sequence of OAV 287,

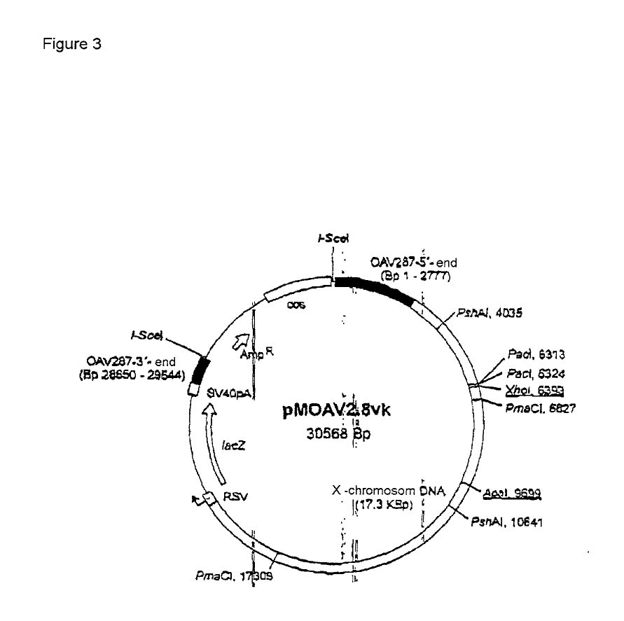

FIG. 3 a schematic representation of the basic vector pMOAV2.8vk for inserting transgenes and subsequently releasing a linear genome of a helper-dependent virus vector by meganuclease digestion.

### EXAMPLES

1. Cloning of the Genome of a Nonhuman Virus into a Cosmid Vector and Characterization of the Location of the Packaging Signal

The cosmid vector pMVKpn was constructed from pMVX-lacZ. The latter vector contains inter alia a bacterial part which is flanked by cleavage sites for meganuclease I-SceI and consists of a bacterial origin of replication (ColE1) and a bacterial ampicillinresistance gene (both from pBluescript, Stratagene), and of the cos element from resistance gene (both from pBluescript, Stratagene) and of the cos element from the cosmid vector pWE15 (Stratagene). To construct pMVKpn, this bacterial part was released from pMVX-lacZ by I-SceI digestion. To construct pMVKpn, a linker which had been obtained by hybridization of the synthetic oligonucleotide SceKpn (5′-CCCTAGGTACCTAGGGATAACAG-3′) was inserted between the I-SceI ends. The effect of the linker in this case was to restore the two I-SceI recognition sequences and to insert a KpnI recognition site between the two restored I-SceI recognition sequences.

The vector pMVKpn contains as important functional element the packaging signal (cos signal) of phage lambda and two directly adjacent cleavage sites for the meganuclease I-SceI with, in between, a unique cleavage site for KpnI. It is possible to insert into the latter by use of highly efficient cosmid cloning methods—packaging of the ligation products into lambda phage heads and infection of E. coli—complete viral genomes of varying origin (for example from infectious viruses) with a size of the order of 25 to 40 KB. The viral genomes cloned in this way can be characterized or manipulated. Infectious, linear genomes with free ITRs can be released from the resulting constructs via the meganuclease cleavage sites. Owing to the length of the recognition sequence (18 Bp), the occurrence of a I-SceI cleavage site in the viral genome can be virtually ruled out.

The genome of the sheep adenovirus OAV287 was inserted into the KpnI cleavage site of pMVKpn via cosmid cloning, resulting in the construct pOAVcos. Transfection of permissive CSL503 cells with meganuclease I-SceI-digested pOAVcos generates efficiently infectious OAV287 (FIG. 1). This construct makes it possible to characterize and manipulate the OAV 287 genome (for example deletions of various regions and/or insertion of transgenes) but also to localize the packaging signal which has not previously been characterized.

To characterize the location of the packaging signal of OAV287, using the cosmid cloning technique various regions of the viral genome were cut out and replaced by spacer DNA and a constitutive lacZ expression cassette.

The constructs were transfected into CSL503 cells, and the cells were then infected with OAV287 as helper virus and lyzed after occurrence of the OAV287-mediated cytopathic effect. Transduction of the lacZ gene after infection of cells with the lyzate indicated that the packaging signal had not been deleted in the particular construct used.

It was possible in this way to localize the location of the packaging signal to the 5′-terminal 2.8 KPb or the 3′-terminal 1 KBp of the viral genome.

2. Production of Basic Vectors for Simplified Insertion of Transgenes into the Helper-dependent Nonhuman Virus Vectors

After partial characterization of the location of the packaging signal of OAV287 (exemplary embodiment 1), a cloning vector was constructed for simplified insertion of transgenes into a helper-dependent sheep adenovirus vector (pMOAV2.8vk). For this purpose, 17.3 kBp of noncoding genomic DNA from the human X chromosome, and a lacZ expression cassette for detection and titration of the derived helper-dependent viruses, were inserted between the 5′-terminal 2.8 kBp and the 3′-terminal 1 kBp of a nonhuman adenovirus.

The X chromosome fragment has two functions: on the one hand, it brings the size of the genome of the helper-dependent sheep virus vector up to the minimum size necessary for packaging in viral capsids and, on the other hand, it provides suitable cleavage sites for inserting transgenes, with or without simultaneous excision of parts of the fragment (insertion site with size adjustment). The basic vector additionally has a cos recognition sequence which makes it possible to employ the cosmid cloning technique for inserting transgenes. In addition, the 3′ and 5′ ends of the sheep adenovirus vector are flanked in the basic vector by cleavage sites for the meganuclease I-SceI, which makes it possible to release linear genomes with terminal ITRs.

To insert the OAV287 genome into pMVKpn, the genome of OAV287 (Genbank Acc No. U40839) was released as KpnI fragment from pOAVpoly (contains wild-type OAV287 genome with a polylinker) and inserted by cosmid cloning into the unique KpnI site of pMVKpn, resulting in pOAVcos.

Subsequently a large part of the OAV genome was replaced by an X chromosome stuffer fragment and an E. coli lacZ expression cassette. By double digestion with Bst1007I and SalI, the OAV genome between Bp 3956 and 28729 was cut out of pOAVcos and replaced by a 26294 Bp fragment which had been released from the vector pMVX-lacZ by SalI/NruI cleavage and comprised 22069 Bp of noncoding human genomic X chromosome DNA (corresponding to Bp 18475-40511 from Genbank Accession No. U82672) and a 4225 Bp expression cassette of the E. coli lacZ gene located at the 5′ terminus under the control of the RSV promoter and the SV40 polyadenylation signal (cosmid cloning). This resulted in the vector pMOAV4.0 in which the two recognition sequences for the meganuclease I-SceI flanked a fragment which is about 32 kBp in total size and in which 3956 Bp from the 5′ end and 896 Bp from the 3′ end of OAV287 flank a human X chromosome fragment and a lacZ expression cassette.

Starting from pMOAV4.0, the 5′ region of OAV was shortened to 2739 Bp by excision of a 1206 Bp PmeI fragment (positions 2739 and 3945 in the OAV genome), resulting in pMOAV2.8. Finally, pMOAV2.8vk was obtained by excision of a XhoI fragment 4757 Bp in size in the X chromosome region of pMOAV2.8 (cf. FIG. 3).

3. Production of a Helper-Dependent Nonhuman Virus Vector with a Transgene

Starting from the basic vectors for generating helper-dependent sheep adenovirus vectors (exemplary embodiment 2), transgene expression cassettes are inserted depending on their number and size in one or more suitable cleavage sites of the cloning site with or without size adjustment by the cosmid cloning technique. Thus, an expression cassette for human alpha-1-antitrypsin was released as XhoI fragment from the plasmid pRSVhAAT described by Hofmann et al., (J. Virol. 73(1999), 6930-6936) and inserted into the XhoI site of pMOAV2.8vk. The total size of the recombinant viral genome remained below 27 kBp. The recombinant viral genome generated in this way was released from the construct by digestion with meganuclease I-SceI and transfected into the helper system. The helper system consists of CSL503 cells as packaging cell line and OAV287 (genome size ˜30 kBp) as helper virus. From the resulting mixture, the helper-dependent sheep adenovirus vector was purified from the helper virus by CsCl density gradient centrifugation—based on differences in density due to different genome sizes.

1. A cloning vector comprising
(a) a packaging sequence of a bacteriophage,
(b) at least one cloning site for inserting heterologous nucleic acid sequences,
(c) at least two cleavage sites for a restriction endonuclease which flank both sides of the cloning site (b),
(d) a bacterial origin of replication, and
(e) a bacterial selection marker gene.

2. A vector as claimed in claim 1, wherein the bacteriophage packaging sequence originates from a lambdoid phage, in particular from phage lambda (cos sequence).

3. A vector as claimed in either of claims 1 or 2, wherein cleavage sites (c) are present for the meganuclease I-SceI.

4. A vector as claimed in any of claims 1 to 3, wherein the selection marker gene (e) comprises an antibiotic-resistance gene.

5. A vector as claimed in any of claims 1 to 4, which comprises inserted into the cloning site (b) the genome of a nonhuman adenovirus.

6. The use of a cloning vector as claimed in any of claims 1 to 5 for producing partially deleted genomes of nonhuman adenoviruses.

7. The use as claimed in claim 6 for identifying and characterizing the packaging sequences of nonhuman adenoviruses.

8. The use as claimed in claim 6 or 7, wherein
(a) a predetermined region of the viral genome inserted into the cloning vector is deleted and replaced by a heterologous nucleic acid comprising a reporter gene cassette,
(b) the constructs produced in (a) are introduced into a helper system which provides the gene products necessary for replication and packaging of the nonhuman adenovirus,
(c) it is determined whether the system according to (b) results in nonhuman adenovirus particles containing the reporter gene, and
(d) steps (a), (b) and (c) are repeated, if necessary, until the cis sequences of the viral genome which are necessary besides the inverted terminal repeats (ITRs) for packaging (packaging sequences) are characterized.

9. The use as claimed in claim 6 for producing a basic vector.

10. A basic vector, in particular for producing a viral vector for gene transfer, comprising
(a) a packaging sequence of a bacteriophage,
(b) viral sequences comprising two inverted terminal repeats (ITRs) and one or more packaging sequences of a nonhuman adenovirus, where the viral sequences are not able to bring about helper-independent viral replication and packaging in a permissive cell line,
(c) a cloning site for insertion of heterologous DNA and, where appropriate, a reporter gene cassette, which are located inside the viral sequences (b),
(d) at least two cleavage sites for a restriction endonuclease which flank both sides of the viral sequences (b),
(e) a bacterial origin of replication and
(f) a bacterial selection marker gene, where sequences (a), (e) and (f) are located outside the sequence region flanked by the cleavage sites (d).

11. A vector as claimed in claim 10, which comprises a reporter gene cassette within the cleavage sites (d).

12. A vector as claimed in claim 10 or 11, wherein the cloning site (c) comprises a heterologous DNA for size adjustment.

13. A vector as claimed in claim 9 to 12, wherein the heterologous DNA comprises a noncoding genomic mammalian DNA.

14. A vector as claimed in claim 11, wherein the reporter gene cassette comprises the E. coli lacZ gene in expressible form.

15. A vector as claimed in any of claims 10 to 14, which comprises a transgene inserted into the cloning site (c).

16. The use of the vector as claimed in any of claims 10 to 15 for producing a helper-dependent nonhuman adenoviral gene transfer vector.

17. A viral gene transfer vector comprising the coat of a nonhuman adenovirus and genetic material which is packaged therein and which comprises
(a) viral sequences comprising two inverted terminal repeats (ITRs) and one or more packaging sequences of a nonhuman adenovirus,
(b) one or more nucleic acid sequences which code for peptides or polypeptides which are heterologous in relation to the nonhuman adenovirus, in operative linkage to expression control sequences and
(c) where appropriate a noncoding DNA for size adjustment,
where the genetic material is not able to bring about helper-independent viral replication and packaging in a permissive cell line.

18. A vector as claimed in claim 17, wherein the genetic material is free of functional nucleic acid sequences of the nonhuman adenovirus with the exception of the cis elements necessary for replication and packaging in the coat.

19. A vector as claimed in claim 17 or 18, wherein the virus is an adenovirus from a nonhuman species selected from mammals and birds.

20. A vector as claimed in claim 19, wherein the virus is an adenovirus from sheep or cattle.

21. A vector as claimed in claim 20, wherein the adenovirus from sheep is an ovine mastadenovirus or an ovine atadenovirus.

22. A vector as claimed in claim 20 or 21, wherein the adenovirus from sheep is the OAV isolate 287.

23. A vector as claimed in claim 20, wherein the adenovirus from cattle is a bovine mastadenovirus or a bovine atadenovirus.

24. A vector as claimed in any of claims 16 to 23, wherein the genetic material comprises
(a) as viral sequences two inverted terminal repeats (ITRs) and one or more packaging sequences and
(b) up to about 30 kBp of foreign DNA.

25. A vector as claimed in any of claims 16 to 24, wherein the genetic material comprises one or more matrix attachment regions (MAR).

26. A vector as claimed in any of claims 16 to 25, wherein the genetic material contains as backbone non-protein-encoding nucleic acids.

27. Genetic material for packaging in a vector as claimed in any of claims 16 to 26.

28. Genetic material as claimed in claim 27 inserted into a cloning vector as claimed in any of claims 1 to 5 or into a basic vector as claimed in any of claims 10 to 15.

29. A system for producing the viral vectors as claimed in any of claims 16 to 26, comprising
(a) the genetic material as claimed in claim 27 or 28,
(b) a helper system for providing the gene products necessary for replication and for packaging of the vector, and
(c) where appropriate means for obtaining and purifying the vectors.

30. A system as claimed in claim 29, wherein the helper system comprises a ready-made packaging cell line.

31. A system as claimed in claim 30, wherein the packaging cell line provides, constitutively or inducibly, the gene products not encoded by the vector itself for replication and packaging of the vector.

32. A system as claimed in any of claims 29 to 31, wherein the helper system comprises a packaging cell line and a helper virus.

33. A system as claimed in claim 32, wherein the helper virus wholly or partly provides the gene products necessary for replication and packaging of the vector.

34. A system as claimed in claim 32 or 33, wherein the packaging cell line provides the viral gene products necessary for replication and packaging of the vector only partly or not at all.

35. A system as claimed in claim 32 or 33, wherein the helper virus is an adenovirus from a nonhuman species.

36. A system as claimed in claim 35, wherein the helper virus is a sheep adenovirus.

37. A system as claimed in claim 36, wherein the sheep helper virus is an ovine mastadenovirus or an ovine atadenovirus.

38. A system as claimed in claim 36 or 37, wherein the sheep helper virus is the OAV isolate 287.

39. A system as claimed in claim 35, wherein the helper virus is an adenovirus from cattle.

40. A system as claimed in claim 39, wherein the helper virus from cattle is a bovine mastadenovirus or a bovine atadenovirus.

41. A system as claimed in any of claims 32 to 40, wherein the helper virus is partially packaging-inhibited.

42. A system as claimed in claim 41, wherein the packaging sequence of the helper virus is partially deleted.

43. A system as claimed in any of claims 32 to 42, wherein the packaging sequence of the helper virus is flanked by recognition sites for a site-specific recombinase.

44. A system as claimed in claim 39, wherein the packaging cell line expresses a gene for a site-specific recombinase.

45. A system as claimed in claim 43 or 44, wherein the recognition sites for a recombinase are loxP sequences, and the recombinase gene is the gene for Cre recombinase.

46. A system as claimed in any of claims 29 to 45, wherein the means for obtaining and purifying the vector comprise a cesium chloride density gradient centrifugation or/and affinity chromatographic separation.

47. The use of the vector as claimed in any of claims 16 to 26 for transferring genetic material into a target cell.

48. The use as claimed in claim 47 additionally comprising the expression of the genetic material in the target cell.

49. The use as claimed in claim 47 or 48, wherein the target cell is a human cell.

50. The use as claimed in any of claims 47 to 49 for nucleic acid vaccination.

51. The use as claimed in any of claims 47 to 49 for gene therapy.

52. The use as claimed in claim 51 for the therapy of congenital or malignant disorders.

53. The use as claimed in any of claims 47 to 49 for obtaining proteins by overexpression in the target cell.
